(19) United States
(12) Patent Application Publication
(10) Pub. No.: US 2021/0137791 A1
(43) Pub. Date: May 13, 2021

(54) CAPSULE FILLER

(71) Applicants: Joey R. Gonzales, Denver, CO (US); Leitisha Moreno, Denver, CO (US)

(72) Inventors: Joey R. Gonzales, Denver, CO (US); Leitisha Moreno, Denver, CO (US)

(21) Appl. No.: 17/088,664

(22) Filed: Nov. 4, 2020

Related U.S. Application Data
(60) Provisional application No. 62/933,787, filed on Nov. 11, 2019.

(51) Int. Cl. A61J 3/07; A61J 3/00

(52) U.S. Cl. CPC A61J 3/074; A61J 3/002

(57) ABSTRACT

A capsule filler includes one or more controllers that are operable to control one or more movement mechanisms and one or more dispensers in order to fill one or more capsules with one or more substances, such as one or more supplements. The movement mechanism may be operable to move the dispenser in one or more directions to control the position of the dispenser with respect to one or more capsules. The dispenser may be operable to dispense one or more substances into the one or more capsules. In this way, the capsule filler may be operable to measure substances and/or otherwise package and/or otherwise prepare the substances for use.

### CROSS-REFERENCE TO RELATED APPLICATION

This application is a non-provisional patent application of and claims the benefit to U.S. Provisional Patent Application No. 62/933,787, filed Nov. 11, 2019, the disclosure of which is hereby incorporated herein by reference in its entirety.

### FIELD

The described embodiments relate generally to packaging. More particularly, the present embodiments relate to a capsule filler.

### BACKGROUND

People take a variety of different supplements and/or other medications or similar products. Such supplements may include nutritional supplements like fish oil and/or other substances. In many cases, these supplements may be stored within a container, such as a capsule, for ease of use.

In some cases, people may obtain such supplements and/or other medications or similar products already pre-measured for a particular dosage and prepared in capsules and/or other containers. In other cases, people may obtain their own supplements and containers and measure dosages, package, and/or otherwise prepare the supplements for use accordingly.

### SUMMARY

The present disclosure relates to a capsule filler. The capsule filler may include one or more controllers that are operable to control one or more movement mechanisms and one or more dispensers in order to fill one or more capsules with one or more substances, such as one or more supplements. The movement mechanism may be operable to move the dispenser in one or more directions to control the position of the dispenser with respect to one or more capsules. The dispenser may be operable to dispense one or more substances into the one or more capsules. In this way, the capsule filler may be operable to measure substances and/or otherwise package and/or otherwise prepare the substances for use.

In various embodiments, a portable liquid capsule filler includes a platform that defines a dispensing area, carriages positioned on the platform, a gantry moveable along the carriages along a first axis, a dispenser moveable along the gantry along a second axis, and a controller. The controller is operable, in response to received instructions, to move the gantry and the dispenser between capsules positioned in the dispensing area and dispense amounts of a liquid into the capsules using the dispenser.

In some examples, the controller operates the dispenser to dispense a first amount of the liquid into a first capsule and a second amount of the liquid into a second capsule. In various implementations of such examples, the first amount is different from the second amount. In some implementations of such examples, the liquid is a first liquid and the controller operates the dispenser to dispense a second liquid into the first capsule.

In various examples, the liquid is an oil. In some examples, the first axis is perpendicular to the second axis.

In a number of examples, the portable liquid capsule filler further includes a closeable case. In various implementations of such examples, the platform, the carriages, the gantry, the dispenser, and the controller are positioned within a space defined by the closeable case.

In some embodiments, the portable liquid capsule filler includes a first carriage, a second carriage, a gantry coupled to the first carriage and the second carriage, a first motor coupled to a first pulley system, a second motor coupled to a second pulley system, a third motor coupled to a third pulley system, a dispenser coupled to the gantry, and a controller. The controller is operable to move the dispenser along the gantry using the third motor to drive the third pulley system, move the gantry along the first carriage and the second carriage by operating the first motor to drive the first pulley system and the second motor to drive the second pulley system, and dispense amounts of a liquid into capsules positioned between the first carriage and the second carriage using the dispenser.

In various examples, the controller operates the first motor and the second motor synchronously to move the gantry along the first carriage and the second carriage. In some examples, the portable liquid capsule filler further includes a pump and a reservoir and the controller dispenses the amounts of the liquid into the capsules by operating the pump to move the amounts of the liquid from the reservoir out the dispenser. In a number of implementations of such examples, the reservoir is configured to be detached from the portable liquid capsule filler.

In some examples, the dispenser is moveable along a first axis and a second axis and the second axis is perpendicular to the first axis. In various examples, the dispenser is configured to be detached from the gantry for manual operation.

In a number of embodiments, a portable liquid capsule filler system includes an electronic device, a pair of carriages, a gantry moveable along the pair of carriages along a first axis, a dispenser moveable along the gantry along a second axis, and a controller. The controller is operable, in response to instructions received from the electronic device, to move the gantry and the dispenser between capsules positioned in a dispensing area and dispense amounts of a liquid into the capsules using the dispenser.

In various examples, the electronic device is configured to present a graphical user interface for instructing the controller. In some implementations of such examples, the graphical user interface allows a user to specify a first amount of the liquid to dispense into a first capsule and a second amount of the liquid to dispense into a second capsule. In a number of implementations of such examples, the liquid is a first liquid and the graphical user interface further allows the user to specify a second liquid to dispense into the first capsule.

In some examples, the electronic device instructs the controller to dispense the amounts of the liquid into the capsules in accordance with a schedule of different capsules. In a number of examples, the electronic device transmits the instructions to the controller using a Bluetooth communication protocol or a WiFi communication protocol. In various examples, the liquid is a nutritional supplement.

### BRIEF DESCRIPTION OF THE DRAWINGS

The disclosure will be readily understood by the following detailed description in conjunction with the accompanying drawings, wherein like reference numerals designate like structural elements.

FIG. 1 depicts a first example capsule filler system.

FIG. 2A depicts a second example capsule filler system.

FIG. 2B depicts the second example capsule filler system of FIG. 2A configured for manual operation.

FIG. 3 depicts a graphical user interface that may be used to control a capsule filler system, such as the first example capsule filler system of FIG. 1 and/or the second example capsule filler system of FIG. 2A.

FIG. 4 is a flow chart illustrating an example method for operating a dispenser system. This method may be performed by the first example capsule filler system of FIG. 1 and/or the second example capsule filler system of FIG. 2A.

### DETAILED DESCRIPTION

Reference will now be made in detail to representative embodiments illustrated in the accompanying drawings. It should be understood that the following descriptions are not intended to limit the embodiments to one preferred embodiment. To the contrary, it is intended to cover alternatives, modifications, and equivalents as can be included within the spirit and scope of the described embodiments as defined by the appended claims.

The description that follows includes sample systems, methods, apparatuses, and computer program products that embody various elements of the present disclosure. However, it should be understood that the described disclosure may be practiced in a variety of forms in addition to those described herein.

It may be challenging for people at home to obtain their own supplements and containers and measure dosages, package, and/or otherwise prepare the supplements for use accordingly. For example, people may find it difficult to measure the dosages of the supplements precisely, put appropriate dosages of supplements into containers (particularly for messy oils or powders), and so on. People may construct apparatuses to automate and/or otherwise make these tasks easier, but such apparatuses may be complex, non-portable and/or otherwise unsuitable to a home environment, and so on.

The following disclosure relates to a capsule filler. The capsule filler may include one or more controllers that are operable to control one or more movement mechanisms and one or more dispensers in order to fill one or more capsules with one or more substances, such as one or more supplements. The movement mechanism may be operable to move the dispenser in one or more directions to control the position of the dispenser with respect to one or more capsules. The dispenser may be operable to dispense one or more substances into the one or more capsules. In this way, the capsule filler may be operable to measure substances and/or otherwise package and/or otherwise prepare the substances for use.

Although the present disclosure is discussed in the context of a capsule filler for supplements, it is understood that this is an example. In any number of implementations, any kinds of containers may be filled with any kind of different substances. Various configurations are possible and contemplated without departing from the scope of the present disclosure.

These and other embodiments are discussed below with reference to FIGS. 1-4. However, those skilled in the art will readily appreciate that the detailed description given herein with respect to these Figures is for explanatory purposes only and should not be construed as limiting.

FIG. 1 depicts a first example capsule filler system 100. The system 100 may include a capsule filler apparatus 101 communicably connected to an electronic device 102. The electronic device 102 may be operable to communicate with one or more controllers 112 of the capsule filler apparatus 101 in order to control one or more components of the capsule filler apparatus 101.

The controller 112 of the capsule filler apparatus 101 may be operable to control one or more movement mechanisms 103 and one or more dispensers 104 in order to fill one or more capsules 111 with one or more substances, such as one or more supplements. The movement mechanism 103 may be operable to move the dispenser 104 in one or more directions to control the position of the dispenser 104 with respect to the capsules 111. The dispenser 104 may be operable to dispense one or more substances into the capsules 111. In this way, the capsule filler apparatus 101 may be operable to measure substances and/or otherwise package and/or otherwise prepare the substances for use.

For example, the capsule filler apparatus 101 may include a platform 109 that defines a dispenser area 108. A holder 110 that is operative to hold a number of capsules 111 may be positioned in the dispenser area 108. The capsules 111 may each be one half that defines an aperture into which one or more substances may be dispensed before the other half is coupled to seal the respective capsule 111.

Carriages 105A and 105B may be positioned on the platform 109. A gantry 106 to which the dispenser 104 is coupled may be moveably mounted to the carriages 105A and 105B (such as via one or more bearings) such that the movement mechanism 103 may be operable to move the gantry 106 (and thus the dispenser) along the carriages 105A and 105B (forward and/or backward with respect to FIG. 1, or a Y axis). The dispenser 104 may itself be moveably mounted to the gantry 106 (such as via one or more bearings) such that the movement mechanism 103 may move the dispenser 104 along the gantry 106 (left and/or right with respect to FIG. 1, or an X axis. The dispenser 104 may also be moveable up and/or down with respect to the capsules 111 in FIG. 1, or a Z axis, in some implementations.

For example, in some implementations, the movement mechanism 103 may be one or more motors connected to one or more gears that interact with one or more mechanical movement mechanisms to move the gantry 106 and/or the dispenser 104. By way of illustration, the gears may interact with one or more pulley driver/belt systems, rack and pinion systems, and so on. Various configurations are possible and contemplated without departing from the scope of the present disclosure.

The electronic device 102 may communicate with the controller 112 to control the movement mechanism 103 in order to move the gantry 106 and/or the dispenser 104 from an initial position to a position corresponding to one of the capsules 111, dispense an amount of substance into the one of the capsules 111 (such as from a reservoir 107 via a pump or other mechanism), and then move the gantry 106 and/or the dispenser 104 to a next capsule 111. In this way, any number of different capsules 111 may be filled with any particular different amount of one or more substances.

In some examples, the substance may be one or more oils and/or other liquids that may be stored in one or more reservoirs 107 and may be pumped through one or more tubes out one or more dispensing heads of the dispenser 104. In some cases, one dispenser head may be used to dispense different liquids from different reservoirs 107, though in other implementations different dispenser heads may be used. In some examples, the electronic device 102 may store information regarding the substance being dispensed, such as a viscosity of the substance, so that the dispenser 104 may be precisely controlled to account for such characteristics to precisely control an amount dispensed. For example, the dispenser 104 may be controlled to dispense more viscous fluids for longer to dispense the same amount of fluid as less viscous fluids. In other examples, a heater element may be used to reduce the viscosity of the fluid.

In still other examples, the substance may be a powder. In such examples, the dispenser maybe an auger at the bottom of a powder storage bin that turns in order to pull powder from the powder storage bin and dispense it into the capsules 111.

In yet other examples, a capsule end dispenser may be included. Such a capsule end dispenser may couple the other half of the capsule 111 after substance dispensing to close the capsule 111. For example, the capsule end dispenser may include a stack of capsule ends that the capsule end dispenser jams down onto the capsules 111, dispenses down on the capsules 111 using compressed air, and so on.

The electronic device 102 may be any kind of device, such as a desktop computing device, a laptop computing device, a smart phone, a mobile computing device, a wearable device, a tablet computing device, and so on. The electronic device 102 may include a variety of components, such as one or more processing units, one or more non-transitory storage media (which may take the form of, but is not limited to, a magnetic storage medium; optical storage medium; magneto-optical storage medium; read only memory; random access memory; erasable programmable memory; flash memory; and so on), communication components, and so on. For example, the processing unit may execute instructions stored in the storage medium in order to perform various electronic device functions, such as presenting one or more user interfaces for controlling the capsule filler apparatus 101, communicating with the controller 112 (such as wirelessly communicating computer numerical control, or “CNC”, instructions via a Bluetooth™ communication protocol, a WiFi communication protocol, and so on), and so on.

The electronic device 102 may provide a graphical user interface that allows a user to specify a number of capsules, a position where the capsules will be, what substances to dispense in what order, how much substance to dispense, and so on. In this way, a precise amount of one or more substances may be dispensed to particular capsules 111 in a particular order. This may correspond to a supplement schedule (e.g., a schedule of different capsules) for a person, such as where a person is constructing 100 capsules with 0.15 milliliters of fish oil, 50 capsules of 0.16 milliliters of fish oil, and 50 capsules of 0.19 milliliters of fish oil in order to follow a particular dosage schedule. Various configurations are possible and contemplated without departing from the scope of the present disclosure.

FIG. 2A depicts a second example capsule filler system 201. The system 201 may include a closeable case 230 that may be opened for operation and/or closed in order to transport the system 201, which may be portable. The system 201 may also include a platform 209 positioned inside the closeable case 230. The platform 209 may define a dispenser area 208. A holder 210 that is operative to hold a number of capsules 211 may be positioned in the dispenser area 208. A guide 229 may be positioned in the dispenser area 208 to guide placement of the holder 210, define an initial start position for dispensing, and so on.

Carriages 205A and 205B may be positioned on the platform 209. A gantry 206 may be moveably mounted to the carriages 205A and 205B (such as via one or more bearings) such that motors 203B and 203C may be operated (such as synchronously) to turn respective gears 220 that are connected to respective belts 221 with respect to pulleys 223 in order to move the gantry 206 (and thus a dispenser 204) along the carriages 205A and 205B (forward and/or backward with respect to FIG. 2A, or a Y axis). However, it is understood that this is an example. In other implementations, mechanisms other than one or more motors 203B, 203C; gears 220; belts 221; and/or pulleys 223 may be used to move the dispenser gantry 224, such as one or more rack and pinion systems. Various configurations are possible and contemplated without departing from the scope of the present disclosure.

Similarly, the dispenser 204 may be mounted to the gantry 206 via a dispenser gantry 224 (such as via one or more bearings) such that a motor 203A may turn a gear 220 that is connected to a belt 221 with respect to a pulley 223 in order to move the dispenser gantry 224 (and thus the dispenser 204) along the gantry 206 (left and/or right with respect to FIG. 2A, or an X axis. However, it is understood that this is an example. In other implementations, mechanisms other than one or more motors 203, gears 220, belts 221, and/or pulleys 223 may be used to move the dispenser gantry 224, such as one or more rack and pinion systems. Various configurations are possible and contemplated without departing from the scope of the present disclosure.

The dispenser 204 may also be moveable up and/or down with respect to the capsules 211 in FIG. 2A, or a Z axis, via a movement mechanism 203D. As shown, the movement mechanism 203D may be a manual knob that interacts with a rack 227 and pinion 228. However, it is understood that this is an example. In other implementations, other manual and/or automatic mechanisms (such a one or more motors, gears, belts, pulleys, rack and pinion systems, and so on) may be used to move the dispenser 204 without departing from the scope of the present disclosure.

The dispenser 204 may include one or more dispenser heads that are connected to one or more pumps 226 via one or more tubes 225 and/or other conduits. The pump 226 may be connected to one or more reservoirs 207. One or more substances may be stored in the reservoir 207, which may be removably connected to the pump 226. The pump 226 may be controlled to dispense a substance from the reservoir 207 through the tube 225 and out the dispenser head of the dispenser 204 into one or more of the capsules 211.

As such, the motors 203A, 203B, and/or 203C may be controlled to move the gantry 206 along the carriages 205A and 205B and/or the dispenser gantry 224 along the gantry 206 to position the dispenser 204 with respect to one or more of the capsules 211. The dispenser 204 may be moved from an initial position to a position corresponding to one of the capsules 211. The pump 226 and/or the dispenser 204 may be controlled to dispense an amount of a substance into the one of the capsules 211. The motors 203A, 203B, and/or 203C may then be controlled to move the gantry 206 along the carriages 205A and 205B and/or the dispenser gantry 224 along the gantry 206 to move the dispenser 204 to a next capsule 211. In this way, any number of different capsules 211 may be filled with any particular different amount of one or more substances.

In one example, the dispenser 204 may include a motorized mechanism that is operable to move multiple dispenser heads (such as three, four, and so on) up and/or down on the Z axis with respect to the capsules 211. Use of this motorized mechanism may enable more precise dispensing as the motorized mechanism may be used to move a dispenser head closer to a capsule 211 before dispensing and then away from the capsule after dispensing. The multiple dispenser heads may be connected to multiple reservoirs 207 (such as three, four, and so on) via multiple pumps 226 (such as three, four, and so on) and tubes 207 (such as three, four, and so on). The use of the multiple dispenser heads may enable dispensing of different substances (which may be located in different reservoirs 207) and/or the same substance into different capsules 211, whether simultaneously, sequentially, and/or in any other order. For example, three dispenser heads may be used to dispense one or more substances into three different capsules 211 at the same time (whether three capsules 211 that are to be filled with three different substances, three capsules that 211 that are to be filled with the same substance, three capsules that are to be sequentially filled with three different substances each, and/or any other configuration). This may greatly reduce time used to fill the capsules 211, particularly over configurations where multiple substances are to be dispensed using an apparatus with only a single dispenser head and/or reservoir 207 that may require swapping out of substances after a first substance is dispensed and before a second substance can be dispensed. However, it is understood that this is an example and that other components may be used in other configurations for other purposes without departing from the scope of the present disclosure.

The system 100 may also be converted to manual operation. For example, FIG. 2B depicts system 201 of FIG. 2A where the dispenser 204 has been detached from the dispenser gantry 224. A user may then manually move the dispenser 204 to dispense a substance manually. Various configurations are possible and contemplated without departing from the scope of the present disclosure.

FIG. 3 depicts a graphical user interface 340 that may be used to control a capsule filler system, such as the system 100 of FIG. 1 and/or the system 201 of FIG. 2A. The graphical user interface 340 may include a map 350 depicting a grid of different squares 351 that each represent a capsule and/or other container (such as one or more jars, syringes, and so on), positional relationships among the different capsules, amounts of one or more substances to dispense, and so on.

Cursor 352 may indicate the square 351 that may be currently selected for modification. Directional control 353 may be used to move the cursor 352 around the map 350. Increase indicator 354 and/or decrease indicator 355 may be used to increase and/or decrease an amount of substance to dispense for a particular square 351 currently indicated by the cursor 352.

However, it is understood that this is an example. In various implementations, various graphical user interfaces with various configurations of the same, similar, and/or different graphical and/or control elements may be used. Various configurations are possible and contemplated without departing from the scope of the present disclosure.

FIG. 4 is a flow chart illustrating an example method 400 for operating a dispenser system. This method 400 may be performed by the system 100 of FIG. 1 and/or the system 201 of FIG. 2A.

At operation 410, a dispenser system may operate. The dispenser system may be the system 100 of FIG. 1, the system 201 of FIG. 2A, and/or another system and/or apparatus.

At operation 420, the dispenser system may determine whether or not to begin a dispensing sequence. For example, the dispenser system may determine to begin a dispensing sequence upon receipt of according instructions via a controller of the dispenser system. If so, the flow may proceed to operation 430. Otherwise, the flow may return to operation 410 where the dispenser system may continue to operate.

At operation 430, after the dispenser system determines to begin a dispensing sequence, the dispenser system may move to a first capsule into which the dispenser system is to dispense one or more substances. For example, the dispenser system may use one or more movement mechanisms of the dispenser system to move one or more dispensers from an initial position to a first capsule. The flow may then proceed to operation 440.

At operation 440, the dispenser system may dispense an amount of the substance into the first capsule. For example, the dispenser system may operate one or more pumps to pull an amount of one or more oils or other fluids from one or more reservoirs through one or more tubes and out one or more dispenser heads into the first capsule. The flow may then proceed to operation 450.

At operation 450, the dispenser system may determine whether or not the sequence includes dispensing one or more substances into one or more additional capsules. If so, the flow may return to operation 430 where the dispenser system may move to the next capsule in the sequence. Otherwise, the flow may return to operation 410 where the dispenser system may continue to operate.

Although the example method 400 is illustrated and described as including particular operations performed in a particular order, it is understood that this is an example. In various implementations, various orders of the same, similar, and/or different operations may be performed without departing from the scope of the present disclosure.

For example, the method 400 is illustrated and described as including operation 430 where the dispenser system may move to a first capsule at the start of a sequence. However, it is understood that this is an example. In some implementations, the dispenser system may already be positioned at the first capsule at the start of a sequence and may start dispensing one or more substances into the first capsule without first moving. Various configurations are possible and contemplated without departing from the scope of the present disclosure.

In various implementations, a portable liquid capsule filler may include a platform that defines a dispensing area, carriages positioned on the platform, a gantry moveable along the carriages along a first axis, a dispenser moveable along the gantry along a second axis, and a controller. The controller may be operable, in response to received instructions, to move the gantry and the dispenser between capsules positioned in the dispensing area and dispense amounts of a liquid into the capsules using the dispenser.

In some examples, the controller may operate the dispenser to dispense a first amount of the liquid into a first capsule and a second amount of the liquid into a second capsule. In various such examples, the first amount may be different from the second amount. In some such examples, the liquid may be a first liquid and the controller may operate the dispenser to dispense a second liquid into the first capsule.

In various examples, the liquid may be an oil. In some examples, the first axis may be perpendicular to the second axis.

In a number of examples, the portable liquid capsule filler may further include a closeable case. In various such examples, the platform, the carriages, the gantry, the dispenser, and the controller may be positioned within a space defined by the closeable case.

In some implementations, the portable liquid capsule filler may include a first carriage, a second carriage, a gantry coupled to the first carriage and the second carriage, a first motor coupled to a first pulley system, a second motor coupled to a second pulley system, a third motor coupled to a third pulley system, a dispenser coupled to the gantry, and a controller. The controller may be operable to move the dispenser along the gantry using the third motor to drive the third pulley system, move the gantry along the first carriage and the second carriage by operating the first motor to drive the first pulley system and the second motor to drive the second pulley system, and dispense amounts of a liquid into capsules positioned between the first carriage and the second carriage using the dispenser.

In various examples, the controller may operate the first motor and the second motor synchronously to move the gantry along the first carriage and the second carriage. In some examples, the portable liquid capsule filler may further include a pump and a reservoir and the controller dispenses the amounts of the liquid into the capsules by operating the pump to move the amounts of the liquid from the reservoir out the dispenser. In a number of implementations of such examples, the reservoir may be configured to be detached from the portable liquid capsule filler.

In some examples, the dispenser may be moveable along a first axis and a second axis and the second axis may be perpendicular to the first axis. In various examples, the dispenser may be configured to be detached from the gantry for manual operation.

In a number of implementations, a portable liquid capsule filler system may include an electronic device, a pair of carriages, a gantry moveable along the pair of carriages along a first axis, a dispenser moveable along the gantry along a second axis, and a controller. The controller may be operable, in response to instructions received from the electronic device, to move the gantry and the dispenser between capsules positioned in a dispensing area and dispense amounts of a liquid into the capsules using the dispenser.

In various examples, the electronic device may be configured to present a graphical user interface for instructing the controller. In some such examples, the graphical user interface may allow a user to specify a first amount of the liquid to dispense into a first capsule and a second amount of the liquid to dispense into a second capsule. In a number of such examples, the liquid may be a first liquid and the graphical user interface may further allow the user to specify a second liquid to dispense into the first capsule.

In some examples, the electronic device may instruct the controller to dispense the amounts of the liquid into the capsules in accordance with a schedule of different capsules. In a number of examples, the electronic device may transmit the instructions to the controller using a Bluetooth communication protocol or a WiFi communication protocol. In various examples, the liquid may be a nutritional supplement.

As described above and illustrated in the accompanying figures, the present disclosure relates to a capsule filler. The capsule filler may include one or more controllers that are operable to control one or more movement mechanisms and one or more dispensers in order to fill one or more capsules with one or more substances, such as one or more supplements. The movement mechanism may be operable to move the dispenser in one or more directions to control the position of the dispenser with respect to one or more capsules. The dispenser may be operable to dispense one or more substances into the one or more capsules. In this way, the capsule filler may be operable to measure the substances and/or otherwise package and/or otherwise prepare the substances for use.

In the present disclosure, the methods disclosed may be implemented as sets of instructions or software readable by a device. Further, it is understood that the specific order or hierarchy of steps in the methods disclosed are examples of sample approaches. In other embodiments, the specific order or hierarchy of steps in the method can be rearranged while remaining within the disclosed subject matter. The accompanying method claims present elements of the various steps in a sample order, and are not necessarily meant to be limited to the specific order or hierarchy presented.

The described disclosure may be provided as a computer program product, or software, that may include a non-transitory machine-readable medium having stored thereon instructions, which may be used to program a computer system (or other electronic devices) to perform a process according to the present disclosure. A non-transitory machine-readable medium includes any mechanism for storing information in a form (e.g., software, processing application) readable by a machine (e.g., a computer). The non-transitory machine-readable medium may take the form of, but is not limited to, a magnetic storage medium (e.g., floppy diskette, video cassette, and so on); optical storage medium (e.g., CD-ROM); magneto-optical storage medium; read only memory (ROM); random access memory (RAM); erasable programmable memory (e.g., EPROM and EEPROM); flash memory; and so on.

The foregoing description, for purposes of explanation, used specific nomenclature to provide a thorough understanding of the described embodiments. However, it will be apparent to one skilled in the art that the specific details are not required in order to practice the described embodiments. Thus, the foregoing descriptions of the specific embodiments described herein are presented for purposes of illustration and description. They are not targeted to be exhaustive or to limit the embodiments to the precise forms disclosed. It will be apparent to one of ordinary skill in the art that many modifications and variations are possible in view of the above teachings.

What is claimed is:

1. A portable liquid capsule filler, comprising:
a platform that defines a dispensing area;
carriages positioned on the platform;
a gantry moveable along the carriages along a first axis;
a dispenser moveable along the gantry along a second axis; and
a controller operable, in response to received instructions, to:
move the gantry and the dispenser between capsules positioned in the dispensing area; and
dispense amounts of a liquid into the capsules using the dispenser.

2. The portable liquid capsule filler of claim 1, wherein the controller operates the dispenser to dispense:
a first amount of the liquid into a first capsule; and
a second amount of the liquid into a second capsule.

3. The portable liquid capsule filler of claim 2, wherein the first amount is different from the second amount.

4. The portable liquid capsule filler of claim 2, wherein:
the liquid is a first liquid; and
the controller operates the dispenser to dispense a second liquid into the first capsule.

5. The portable liquid capsule filler of claim 1, wherein the liquid is an oil.

6. The portable liquid capsule filler of claim 1, wherein the first axis is perpendicular to the second axis.

7. The portable liquid capsule filler of claim 1, further comprising a closeable case wherein the platform, the carriages, the gantry, the dispenser, and the controller are positioned within a space defined by the closeable case.

8. A portable liquid capsule filler, comprising:
a first carriage;
a second carriage;
a gantry coupled to the first carriage and the second carriage;
a first motor coupled to a first pulley system;
a second motor coupled to a second pulley system;
a third motor coupled to a third pulley system;
a dispenser coupled to the gantry; and
a controller operable to:
move the dispenser along the gantry using the third motor to drive the third pulley system;
move the gantry along the first carriage and the second carriage by operating the first motor to drive the first pulley system and the second motor to drive the second pulley system; and
dispense amounts of a liquid into capsules positioned between the first carriage and the second carriage using the dispenser.

9. The portable liquid capsule filler of claim 8, wherein the controller operates the first motor and the second motor synchronously to move the gantry along the first carriage and the second carriage.

10. The portable liquid capsule filler of claim 8, further comprising:
a pump; and
a reservoir; wherein:
the controller dispenses the amounts of the liquid into the capsules by operating the pump to move the amounts of the liquid from the reservoir out the dispenser.

11. The portable liquid capsule filler of claim 10, wherein the reservoir is configured to be detached from the portable liquid capsule filler.

12. The portable liquid capsule filler of claim 8, wherein:
the dispenser is moveable along a first axis and a second axis; and
the second axis is perpendicular to the first axis.

13. The portable liquid capsule filler of claim 8, wherein the dispenser is configured to be detached from the gantry for manual operation.

14. A portable liquid capsule filler system, comprising:
an electronic device;
a pair of carriages;
a gantry moveable along the pair of carriages along a first axis;
a dispenser moveable along the gantry along a second axis; and
a controller operable, in response to instructions received from the electronic device, to:
move the gantry and the dispenser between capsules positioned in a dispensing area; and
dispense amounts of a liquid into the capsules using the dispenser.

15. The portable liquid capsule filler system of claim 14, wherein the electronic device is configured to present a graphical user interface for instructing the controller.

16. The portable liquid capsule filler system of claim 15, wherein the graphical user interface allows a user to specify:
a first amount of the liquid to dispense into a first capsule; and
a second amount of the liquid to dispense into a second capsule.

17. The portable liquid capsule filler system of claim 16, wherein:
the liquid is a first liquid; and
the graphical user interface further allows the user to specify a second liquid to dispense into the first capsule.

18. The portable liquid capsule filler system of claim 14, wherein the electronic device instructs the controller to dispense the amounts of the liquid into the capsules in accordance with a schedule of different capsules.

19. The portable liquid capsule filler system of claim 14, wherein the electronic device transmits the instructions to the controller using a Bluetooth communication protocol or a WiFi communication protocol.

20. The portable liquid capsule filler system of claim 14, wherein the liquid is a nutritional supplement.
